(19) United States
(12) Patent Application Publication
(10) Pub. No.: US 2026/0060735 A1
(43) Pub. Date: Mar. 5, 2026

(54) TISSUE CONTACT ASSESSMENT WITH COMBINED MAPPING AND ABLATION CATHETER

(71) Applicant: Boston Scientific Scimed, Inc., Maple Grove, MN (US)

(72) Inventors: Brendan Early Koop, Ham Lake, MN (US); Kara Elizabeth Garrott, Minneapolis, MN (US); Megan Wurden, Waltham, MA (US)

(21) Appl. No.: 19/310,406

(22) Filed: Aug. 26, 2025

Related U.S. Application Data
(60) Provisional application No. 63/688,237, filed on Aug. 28, 2024.

(51) Int. Cl. A61B 18/00; A61B 5/00; A61B 5/287; A61B 5/339

(52) U.S. Cl. CPC A61B 18/00; A61B 5/287; A61B 5/339; A61B 5/6858; A61B 5/6859; A61B 5/6869; A61B 2018/0016; A61B 2018/00351; A61B 2018/00577; A61B 2018/00613; A61B 2018/00839; A61B 2018/00875; A61B 2018/00892; A61B 2560/0468; A61B 2562/046

(57) ABSTRACT

A system for ablating cardiac tissue through irreversible electroporation is disclosed. The system includes an electroporation and mapping catheter, a graphical display, and a controller coupled to the graphical display and the catheter. The catheter includes an electrode assembly defining a distally located central hub portion and splines, each spline including a distal end portion extending from the central hub portion, and a proximal end portion attached to and constrained by an outer shaft. The electrode assembly includes electrodes having sensing electrodes located on each spline. The controller determines an impedance from a four-terminal configuration wherein a current is injected to a current carrying pair of electrodes and a voltage is measured across a sensing pair of electrodes comprising sensing electrodes. The controller generates a graphical representation of a model of the impedance of the spline on the graphical display.

### CROSS REFERENCE TO RELATED APPLICATIONS

This application claims priority to U.S. Provisional Patent Application No. 63/688,237 entitled “TISSUE CONTACT ASSESSMENT WITH COMBINED MAPPING AND ABLATION CATHETER,” filed Aug. 28, 2024, which is hereby incorporated by reference in its entirety.

### TECHNICAL FIELD

The present disclosure relates to medical systems and methods for ablating tissue in a patient. More specifically, the present disclosure relates to medical systems and methods for ablation of tissue by electroporation.

### BACKGROUND

Ablation procedures are used to treat many different conditions in patients. Ablation can be used to treat cardiac arrhythmias, benign tumors, cancerous tumors, and to control bleeding during surgery. Usually, ablation is accomplished through thermal ablation techniques including radio-frequency (RF) ablation and cryoablation. In RF ablation, a probe is inserted into the patient and radio frequency waves are transmitted through the probe to the surrounding tissue. The radio frequency waves generate heat, which destroys surrounding tissue and cauterizes blood vessels. In cryoablation, a hollow needle or cryoprobe is inserted into the patient and cold, thermally conductive fluid is circulated through the probe to freeze and kill the surrounding tissue. RF ablation and cryoablation techniques indiscriminately kill tissue through cell necrosis, which may damage or kill otherwise healthy tissue, such as tissue in the esophagus, phrenic nerve cells, and tissue in the coronary arteries.

Another ablation technique uses electroporation. In electroporation, or electro-permeabilization, an electrical field is applied to cells to increase the permeability of the cell membrane. The electroporation can be reversible or irreversible, depending on the strength of the electric field. If the electroporation is reversible, the increased permeability of the cell membrane can be used to introduce chemicals, drugs, and/or deoxyribonucleic acid (DNA) into the cell, prior to the cell healing and recovering. If the electroporation is irreversible, the affected cells are killed through apoptosis.

Irreversible electroporation can be used as a nonthermal ablation technique. In irreversible electroporation, trains of short, high voltage pulses are used to generate electric fields that are strong enough to kill cells through apoptosis. In ablation of cardiac tissue, irreversible electroporation can be a safe and effective alternative to the indiscriminate killing of thermal ablation techniques, such as RF ablation and cryoablation. Irreversible electroporation can be used to kill targeted tissue, such as myocardium tissue, by using an electric field strength and duration that kills the targeted tissue but does not permanently damage other cells or tissue, such as non-targeted myocardium tissue, red blood cells, vascular smooth muscle tissue, endothelium tissue, and nerve cells. There is a continuing need for improved devices and methods for performing cardiac tissue ablation through irreversible electroporation.

### SUMMARY

In Example 1, a system for ablating cardiac tissue through irreversible electroporation, the system comprising: an electroporation and mapping catheter for performing irreversible electroporation, the catheter comprising: a shaft having a proximal end and an opposite distal end; and an electrode assembly extending distally from the distal end of the shaft, the electrode assembly defining a distally located central hub portion and a plurality of splines each including a distal end portion extending from the central hub portion, and a proximal end portion attached to and constrained by the outer shaft, the electrode assembly having an expanded configuration and a collapsed configuration, the electrode assembly comprising a plurality of electrodes including: a distal ablation electrode including an ablation electrode hub portion located on the distal hub portion, and a plurality of radial segments integrally formed with the ablation electrode hub portion, each of the radial segments extending proximally along a portion of a respective one of the splines and terminating in a proximal end; and a set of spline sensing electrodes including a plurality of sensing electrodes located on each spline; a graphical display; and a controller coupled to the graphical display and the catheter, the controller configured to: determine an impedance from a four-terminal configuration, wherein a current is injected to a current carrying pair of the plurality of electrodes and a voltage is measured across a sensing pair of electrodes comprising a plurality of sensing electrodes; and generate, on the graphical display, a graphical representation of a model of the impedance of the spline.

In Example 2, the system of Example 1, wherein the central hub portion includes a hub sensing electrode.

In Example 3, the system of Example 2, wherein the current carrying pair of electrodes includes the hub sensing electrode and a sensing electrode on a spline of the plurality of splines.

In Example 4, the system of any of Examples 1 to 3, wherein the shaft includes a shaft electrode.

In Example 5, the system of Example 4, wherein the current carrying pair of electrodes includes the ablation electrode and the shaft electrode.

In Example 6, the system of any of Example 1 to 5, wherein the plurality of splines define an inner space in the expanded configuration, and the electrode assembly includes a post electrode disposed in the inner space.

In Example 7, the system of Example 6, wherein the current carrying pair of electrodes includes the post electrode.

In Example 8, the system of any of Examples 1 to 7, the catheter further comprising a plurality of proximal ablation electrodes, each of the proximal ablation electrodes located on a respective one of the spline and having a distal end spaced from the proximal end of the adjacent radial segment of the distal ablation electrode.

In Example 9, the system of any of Examples 1 to 8, wherein the impedance is determined for a spline from the voltage measured across the sensing pair of electrodes comprising a pair of adjacent sensing electrodes on the spline.

In Example 10, the system of Example 9, wherein the graphical representation includes a color from a plurality of colors representative of impedance associated with that spline.

In Example 11, the system of any of Examples 1 to 10, wherein the impedance is determined from multiplexing multiple sets of sensing pairs of electrodes.

In Example 12, the system of Example 11, wherein the plurality of electrodes of a sensing pair of the multiple sets of sensing pairs includes electrodes on adjacent splines.

In Example 13, the system of any of Examples 1-12, wherein the graphical representation includes a vector from a plurality of vectors representative of impedance associated with each of the sensing electrodes of the plurality of sensing electrodes.

In Example 14, the system of any of Examples 1 to 9 and 11 to 13, wherein the graphical representation includes a color from a plurality of colors representative of impedance associated with each of the sensing electrodes of the plurality of sensing electrodes.

In Example 15, the system of Example 1, wherein the determined impedance is applied to generate a map of the heart.

In Example 16, a system for ablating cardiac tissue through irreversible electroporation, the system comprising: an electroporation and mapping catheter for performing irreversible electroporation, the catheter comprising: a shaft having a proximal end and an opposite distal end; and an electrode assembly extending distally from the distal end of the shaft, the electrode assembly defining a distally located central hub portion and a plurality of splines each including a distal end portion extending from the central hub portion, and a proximal end portion attached to and constrained by the outer shaft, the electrode assembly having an expanded configuration and a collapsed configuration, the electrode assembly comprising a plurality of electrodes including: a distal ablation electrode including an ablation electrode hub portion located on the distal hub portion, and a plurality of radial segments integrally formed with the ablation electrode hub portion, each of the radial segments extending proximally along a portion of a respective one of the splines and terminating in a proximal end; and a set of spline sensing electrodes including a plurality of sensing electrodes located on each spline; a graphical display; and a controller coupled to the graphical display and the catheter, the controller configured to: determine an impedance from a four-terminal configuration, wherein a current is injected to a current carrying pair of the plurality of electrodes and a voltage is measured across a sensing pair of electrodes comprising a plurality of sensing electrodes; and generate, on the graphical display, a graphical representation of a model of the impedance of the spline, wherein the graphical representation includes a color from a plurality of colors representative of impedance associated with each of the sensing electrodes of the plurality of sensing electrodes.

In Example 17, the system of Example 16, wherein the central hub portion includes a hub sensing electrode.

In Example 18, the system of Example 17, wherein the current carrying pair of electrodes includes the hub sensing electrode and a sensing electrode on a spline of the plurality of splines.

In Example 19, the system of Example 16, wherein the shaft includes a shaft electrode.

In Example 20, the system of Example 19, wherein the current carrying pair of electrodes includes the ablation electrode and the shaft electrode.

In Example 21, the system of Example 16, wherein the plurality of splines define an inner space in the expanded configuration, and the electrode assembly includes a post electrode disposed in the inner space.

In Example 22, the system of Example 21, wherein the current carrying pair of electrodes includes the post electrode.

In Example 23, the system of Example 16, the catheter further comprising a plurality of proximal ablation electrodes, each of the proximal ablation electrodes located on a respective one of the spline and having a distal end spaced from the proximal end of the adjacent radial segment of the distal ablation electrode.

In Example 24, the system of Example 16, wherein the impedance is determined for a spline from the voltage measured across the sensing pair of electrodes comprising a pair of adjacent sensing electrodes on the spline.

In Example 25, the system of Example 24, wherein the graphical representation includes a color from a plurality of colors representative of impedance associated with that spline.

In Example 26, the system of Example 16, wherein the impedance is determined from multiplexing multiple sets of sensing pairs of electrodes.

In Example 27, the system of Example 16, wherein the graphical representation includes a vector from a plurality of vectors representative of impedance associated with each of the sensing electrodes of the plurality of sensing electrodes.

In Example 28, the system of Example 16, wherein the graphical representation includes a color from a plurality of colors representative of impedance associated with each of the sensing electrodes of the plurality of sensing electrodes.

In Example 29, a system for ablating cardiac tissue through irreversible electroporation, the system comprising: an electroporation and mapping catheter for performing irreversible electroporation, the catheter comprising: a shaft having a proximal end and an opposite distal end; and an electrode assembly extending distally from the distal end of the shaft, the electrode assembly defining a distally located central hub portion and a plurality of splines each including a distal end portion extending from the central hub portion, and a proximal end portion attached to and constrained by the outer shaft, the electrode assembly having an expanded configuration and a collapsed configuration, the electrode assembly comprising a plurality of electrodes including: a distal ablation electrode including an ablation electrode hub portion located on the distal hub portion, and a plurality of radial segments integrally formed with the ablation electrode hub portion, each of the radial segments extending proximally along a portion of a respective one of the splines and terminating in a proximal end; and a set of spline sensing electrodes including a plurality of sensing electrodes located on each spline; a graphical display; and a controller coupled to the graphical display and the catheter, the controller configured to: determine an impedance from a four-terminal configuration, wherein a current is injected to a current carrying pair of the plurality of electrodes and a voltage is measured across a sensing pair of electrodes comprising a plurality of sensing electrodes; and generate, on the graphical display, a graphical representation of a model of the impedance of that spline, wherein the graphical representation includes a color from a plurality of colors representative of impedance associated with each of the sensing electrodes of the plurality of sensing electrodes.

In Example 30, the system of Example 29, wherein the impedance is determined from multiplexing multiple sets of sensing pairs of electrodes.

In Example 31, the system of Example 30, wherein the plurality of electrodes of a sensing pair of the multiple sets of sensing pairs includes electrodes on adjacent splines.

In Example 32, the system of Example 31, wherein the graphical representation includes a vector from a plurality of vectors representative of impedance associated with each of the sensing electrodes of the plurality of sensing electrodes.

In Example 33, a system for ablating cardiac tissue through irreversible electroporation, the system comprising: an electroporation and mapping catheter for performing irreversible electroporation, the catheter comprising: a shaft having a proximal end and an opposite distal end; and an electrode assembly extending distally from the distal end of the shaft, the electrode assembly defining a distally located central hub portion and a plurality of splines each including a distal end portion extending from the central hub portion, and a proximal end portion attached to and constrained by the outer shaft, the electrode assembly having an expanded configuration and a collapsed configuration, the electrode assembly comprising a plurality of electrodes including: a distal ablation electrode including an ablation electrode hub portion located on the distal hub portion, and a plurality of radial segments integrally formed with the ablation electrode hub portion, each of the radial segments extending proximally along a portion of a respective one of the splines and terminating in a proximal end; and a set of spline sensing electrodes including a plurality of sensing electrodes located on each spline; a graphical display; and a controller coupled to the graphical display and the catheter, the controller configured to: determine an impedance from a four-terminal configuration, wherein a current is injected to a current carrying pair of the plurality of electrodes and a voltage is measured across a sensing pair of electrodes comprising a plurality of sensing electrodes; and generate, on the graphical display, a graphical representation of a model of the impedance of the spline, wherein the graphical representation includes a color from a plurality of colors representative of impedance associated with each of the sensing electrodes of the plurality of sensing electrodes, and wherein the graphical representation includes a vector from a plurality of vectors representative of impedance associated with each of the sensing electrodes of the plurality of sensing electrodes.

In Example 34, the system of Example 33, wherein the determined impedance is applied to generate a map of the heart.

In Example 35, the system of Example 33, wherein the impedance is determined from multiplexing multiple sets of sensing pairs of electrodes.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for treating a heart of the patient, using an example electrophysiology system.

FIG. 2A is a perspective illustration of a distal portion of a splined catheter for use in the example electrophysiology system of FIG. 1.

FIGS. 2B-2C are partial plan views an electrode assembly of the splined catheter of FIG. 2A shown in two-dimensions.

FIG. 3A is a partial perspective view illustrating another electrode assembly of a splined catheter for use in the example electrophysiology system of FIG. 1.

FIG. 3B is a partial perspective view of the electrode assembly of FIG. 3A illustrating an example to determine local impedances with a four-terminal configuration.

FIG. 3C is a partial perspective view of the electrode assembly of FIG. 3A illustrating another example to determine local impedances with a four-terminal configuration.

FIG. 3D is a partial perspective view of the electrode assembly of FIG. 3A illustrating still another example to determine local impedances with a four-terminal configuration.

FIG. 4 is a schematic diagram of an example controller for use with the example electrophysiology system of FIG. 1.

FIG. 5 is a flow diagram illustrating an example configuration of the example controller of FIG. 4.

FIGS. 6A-6D are schematic diagrams illustrating example graphical representations generated with the example configuration of FIG. 5.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

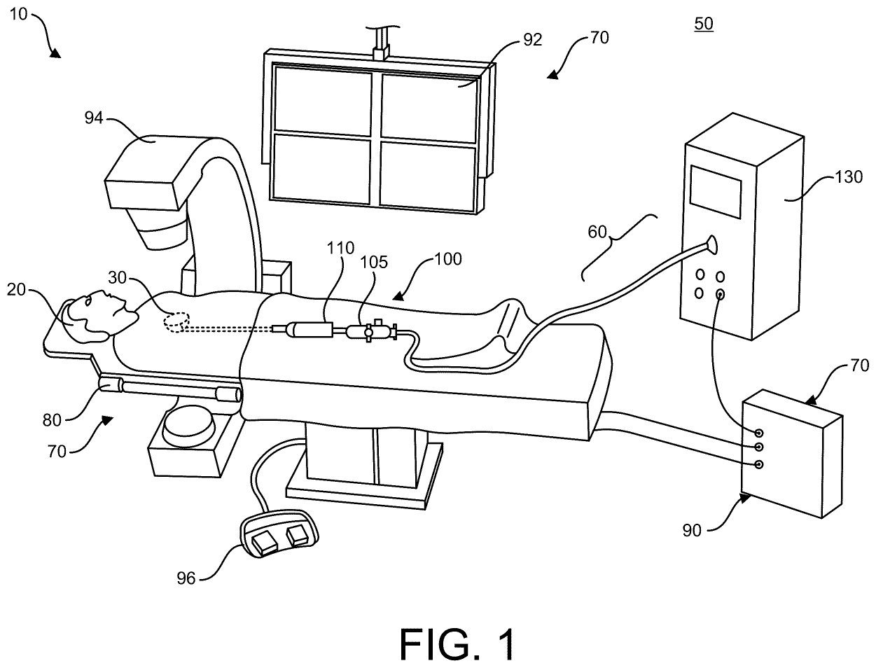

FIG. 1 illustrates an example clinical setting 10 for treating a patient 20, such as for treating a heart 30 of the patient 20, using an electrophysiology system 50, in accordance with the disclosure. The electrophysiology system 50 includes an electroporation catheter system 60 and an electro-anatomical mapping (EAM) system 70. The example electroporation catheter system 60 includes an electroporation catheter 105, an introducer sheath 110, and an electroporation console 130. Additionally, the electroporation catheter system 60 includes various connecting elements, such as cables, that operably connect the components of the electroporation catheter system 60 to one another and to the components of the EAM system 70. In general, the EAM mapping system 70 includes a localization field generator 80, a mapping and navigation controller 90, and a display 92. Also, the clinical setting 10 can include additional equipment such as imaging equipment 94 (represented by the C-arm) and various controller elements, such as a foot controller 96, configured to allow an operator to control various aspects of the electrophysiology system 50. The clinical setting 10 may have other components and arrangements of components that are not shown in FIG. 1. Other arrangements of connecting elements, including wireless connecting elements, are contemplated.

The electroporation catheter system 60 is configured to deliver electric field energy to targeted tissue in the patient's heart 30 to create cell death in tissue, for example, rendering the tissue incapable of conducting electrical signals. Also, the electroporation catheter system 60 is configured to generate, based on models of electric fields, graphical representations of the electric fields that can be produced using the electroporation catheter 105 and to overlay, on the display 92, the graphical representations of the electric fields or expected or predicted lesions on an anatomical map of the patient's heart to aid a user in planning ablation by irreversible electroporation using the electroporation catheter 105 prior to delivering energy. In embodiments, the electroporation catheter system 60 is configured to generate the graphical representations of the electric fields based on characteristics of the electroporation catheter 105 and the position of the electroporation catheter 105 in the patient 20, such as in the heart 30 of the patient 20. The electroporation catheter system 60 is configured to generate the graphical representations of the electric fields based on characteristics of the electroporation catheter 105 and the position of the electroporation catheter 105 in the patient 20, such as in the heart 30 of the patient 20, and the characteristics of the tissue surrounding the catheter 105, such as measured impedances of the tissue.

The introducer sheath 110 is operable to provide a delivery conduit through which the electroporation catheter 105 can be deployed to the specific target sites within the patient's heart 30. Access to the patient's heart can be obtained through a vessel, such as a peripheral artery or vein. Once access to the vessel is obtained, the electroporation catheter 105 can be navigated to within the patient's heart, such as within a chamber of the heart.

The example electroporation catheter 105 includes an elongated catheter shaft and distal end configured to be deployed proximate target tissue, such as within a chamber of the patient's heart. The distal end includes an electrode assembly to effect treatment. The catheter 105 is capable of being formed into a plurality of configurations. For example, if the distal end region of the catheter is within the patient's vasculature or is within a sheath as a catheter assembly, such as to travel to the patient to the chamber of the heart, the electrode assembly is in a collapsed configuration to fit within the sheath. Once the catheter has reached the destination in the chamber of the heart, for example, or the sheath is retracted from the distal region of the catheter 105 (or the shaft catheter is extended past the sheath), and the electrode assembly is arranged in an expanded configuration for use. In one embodiment, the electrode assembly can assume other configurations, such as an intermediate configuration between the collapsed and expanded configurations, such as an additional use configuration.

The electrode assembly includes an electrode assembly comprising a plurality of electrodes. For example, the electrode assembly includes a plurality of spaced-apart electrodes or multiple spaced-apart sets or groups of spaced-apart electrodes. In some examples, an electrode, such as a plurality of spaced-apart electrodes, can be deployed on the catheter shaft in addition to electrodes on the electrode assembly. In one example, the plurality of electrodes can be formed of a conductive, solid-surface, biocompatible material and are spaced-apart across electrical insulators. Each of the plurality of electrodes is electrically coupled to an associated elongated lead conductor that extend along the shaft to a catheter proximal end. In one example, each electrode of the spaced-apart electrodes corresponds with a separate, single lead conductor. In another example, a plurality of electrodes may be coupled to a single lead conductor. Other configurations are contemplated. The plurality of lead conductors can be electrically insulated from one another within an electrically insulating sheath along the catheter shaft, such as with an electrically insulating polymer sheath. The lead conductors can be electrically coupled to plug in the proximal region of the electroporation catheter 105, such as a plug configured to be mechanically and electrically coupled to the electroporation console 130 and the EAM system 70, for example, either directly or via intermediary electrical conductors such as cabling.

The electrode assemblies and associated electrodes are configured for, among other things, sensing cardiac electrical signals, ablation, localization of the electrode assembly within the patient anatomy such as via the EAM system 70, signal reference, and to determine proximity to target tissue within the anatomy. In some embodiments, the catheter 105 is configured for cardiac mapping, and the electrodes are sensing, or mapping, electrodes configured to be used to collect physiological (electrical) signals to be used to generate electroanatomical maps. An example of a physiological signal that the sensing electrode can acquire includes an intracardiac electrogram (ECG) signal. In some embodiments, the catheter 105 can be a mapping and ablation catheter, and the electrodes can include ablation electrodes, or an ablation electrode assembly, that are configured to deliver ablation electric field energy and sensing electrodes, or a sensing electrode assembly, for mapping purposes. The ablation electrodes in embodiments of an electroporation catheter are configured to receive pulsed electrical signals or waveforms from the console 130 and create pulsed electric fields sufficient to ablate target tissue via irreversible electroporation. The sensing electrodes in the electrode assembly can be electrically coupled to a one or more lead conductors that extends the length of the shaft that are configured to carry an electrical signal received at the sensing electrode. In some examples, an electrode in the electrode assembly can be configured to only perform an ablation or the electrode in the electrode assembly can be configured to only perform mapping. In some examples, an electrode can operate as an ablation electrode in an ablation mode of the electrophysiology system 50 and as a sensing electrode in a sensing or mapping mode of the system 50. Some examples of mapping and ablation catheters are smaller in profile or in the volume of the electrode assembly than catheters that just perform mapping, and clinicians can map a given location within the heart with fewer passes across the chamber with mapping catheters than with mapping and ablation catheters.

In one example, the electroporation console 130 is configured to provide an electrical signal, such as a plurality of concurrent or space-apart-time electrical signals, to the electrically connected electroporation catheter 105 along lead conductors to the spaced-apart electrodes. The spaced-apart electrodes are configured to generate a selected electrical field proximate the target tissue, based on the electrical signals from the electroporation console 130, to effect electroporation.

Embodiments of the present disclosure provide systems, devices, and methods for selective and rapid application of pulsed electric fields to ablate tissue by irreversible electroporation. Generally, the systems, devices, and methods described herein may be used to generate large electric field magnitudes at desired regions of interest and reduce peak electric field values elsewhere in order to reduce unnecessary tissue damage and electrical arcing. An irreversible electroporation system as described herein may include a signal generator and a processor configured to apply one or more voltage pulse waveforms to a selected set of electrodes of an ablation device to deliver energy to a region of interest (e.g., ablation energy for a set of tissue in a pulmonary vein ostium or antrum). The pulse waveforms disclosed herein may aid in therapeutic treatment of a variety of cardiac arrhythmias (e.g., atrial fibrillation). In order to deliver the pulse waveforms generated by the signal generator, one or more electrodes of the ablation device may have an insulated electrical lead configured for sustaining a voltage potential in the order of several hundred volts to several thousand volts. The electrodes may be independently addressable such that each electrode may be controlled (e.g., deliver energy) independently of any other electrode of the device. In this manner, the electrodes may deliver different energy waveforms with different timing synergistically for electroporation of tissue.

Pulse waveforms for electroporation energy delivery as disclosed herein may enhance the safety, efficiency and effectiveness of energy delivery to tissue by reducing the electric field threshold associated with irreversible electroporation, thus yielding more effective ablative lesions with a reduction in total energy delivered. In some embodiments, the voltage pulse waveforms disclosed herein may be hierarchical and have a nested structure. For example, the pulse waveform may include hierarchical groupings of pulses having associated timescales. In some embodiments, the methods, systems, and devices disclosed herein may comprise one or more of the methods, systems, and devices described in International Application Serial No. PCT/US2016/057664, filed on Oct. 19, 2016, and titled “SYSTEMS, APPARATUSES AND METHODS FOR DELIVERY OF ABLATIVE ENERGY TO TISSUE,” the contents of which are hereby incorporated by reference in its entirety.

A selected electrical field can be generated with the electrodes configured as ablation electrodes to effect electroporation. A first ablation electrode, or first group of ablation electrodes, can be selected to be an anode and a different, second ablation electrode, or second group of ablation electrodes, can be selected to be a cathode, such that electrical fields can be generated between the anode and cathode based on signals, such as pulses, provided to the ablation electrodes from the electroporation console 130. The console 130 provides electric pulses of different lengths and magnitudes to the ablation electrodes on the catheter 105. The electric pulses can be provided in a continuous stream of pulses or in multiple, separate trains of pulses. Pulse parameters of interest include the number of pulses, the duty cycle of the pulses, the spacing of pulse trains, the voltage or magnitude of the pulses including the peak voltages, and the duration of the voltages. For example, the console 130 can select two or more ablation electrodes of the electrode assembly and provides pulses to the selected electrodes to generate electric fields between the selected electrodes to provide pulsed field ablation (PFA). For example, PFA can be performed with monophasic waveforms and biphasic waveforms. Without being bound to a particular theory, electric field strengths in the range of generally 200-250 volts per centimeter (V/cm) with microsecond-scale pulse duration have been demonstrated to provide reversible electroporation in cardiac tissue. Electric field strengths at approximately 400 V/cm have been demonstrated to provide irreversible electroporation in cardiac tissue of interest, such as targeted myocardium tissue and endocardium tissue, with demonstrable sparing of red blood cells, vascular smooth muscle tissue, endothelium tissue, nerves and other non-targeted proximate tissue.

Additionally, the electrode assembly on catheter 105 can be operated in a selected mode such as monopolar mode or bipolar mode. During monopolar operation of the catheter 105, an ablation electrode, a group of ablation electrodes, or the entire electrode assembly are configured as one of an anode or a cathode. None of the electrodes in the electrode assembly are configured as a the other of the cathode or the anode. Instead, the other of the cathode or the anode is provided in the form of a pad dispersive electrode located on the patient, typically on the back, buttocks, or other suitable anatomical location during electroporation. An electrical field is formed between an activated electrode of the electrode assembly and the pad dispersive electrode. During bipolar operation of the catheter 105, a first set of one or more electrodes of the electrode assembly, is configured as the anode and a second set of one or more electrodes of the electrode assembly, is configured as the cathode, to generate the electric field. In this example, a pad dispersive electrode is not used, and the electrical field is not extended in the patient's body, but rather through a localized portion of tissue proximate the electrode assembly. For example, the electrodes on the ablation electrode assembly are configured as the one of the anode or cathode and electrodes on the shaft proximate the distal end are configured as the other of the cathode or anode.

The EAM system 70 is operable to track the location of the various components of the electroporation catheter system 60, and to generate high-fidelity three-dimensional anatomical and electro-anatomical maps of the heart, including portions of the heart such as cardiac chambers of interest or other structures of interest such as the sinoatrial node or atrioventricular node. In one illustrative example, the EAM system 70 can include the RHYTHMIA™ HDx mapping system marketed by Boston Scientific Corporation. Also, the mapping and navigation controller 90 of the EAM system 70 includes one or more controllers, such as microprocessors or computers, that execute code out of memory to control or perform functional aspects of the EAM system 70, in which the memory, can be part of the one or more controllers, microprocessors, computers, or part of a memory device accessible through a computer network.

The EAM system 70 generates a localization field, via the field generator 80, to define a localization volume about the heart 30, and a location sensor or sensing element on a tracked device, such as sensors on the electroporation catheter 105, generate an output that can be processed by the mapping and navigation controller 90 to track the location of the sensor, and consequently, the corresponding device, within the localization volume. In the illustrated example, the device tracking is accomplished using magnetic tracking techniques, in which the field generator 80 is a magnetic field generator that generates a magnetic field defining the localization volume, and location sensors on the tracked devices are magnetic field sensors.

In other examples, impedance tracking methodologies may be employed to track the locations of the various devices. In such examples, the localization field is an electric field generated, for example, by an external field generator arrangement, such as surface electrodes, by intra-body or intra-cardiac devices, such as an intracardiac catheter, or both. In these examples, the location sensing elements can constitute electrodes on the tracked devices that generate outputs received and processed by the mapping and navigation controller 90 to track the location of the various location sensing electrodes within the localization volume.

The EAM system 70 can be equipped for both magnetic and impedance tracking capabilities. In such examples, impedance tracking accuracy can, in some instances be enhanced by first creating a map of the electric field induced by the electric field generator within the cardiac chamber of interest using a probe equipped with a magnetic location sensor, as is possible using the RHYTHMIA HDx™ mapping system. One exemplary probe is the INTELLAMAP ORION™ mapping catheter marketed by Boston Scientific Corporation.

Regardless of the tracking methodology employed, the EAM system 70 utilizes the location information for the various tracked devices, along with cardiac electrical activity acquired by, for example, the electroporation catheter 105 or another catheter or probe equipped with sensing electrodes, to generate, and display via the display 92, detailed three-dimensional geometric anatomical maps or representations of the heart tissue and voids such as cardiac chambers as well as electro-anatomical maps in which cardiac electrical activity of interest is superimposed on the geometric anatomical maps. Furthermore, the EAM system 70 can generate a graphical representation of the various tracked devices within the geometric anatomical map or the electro-anatomical map.

Each cardiac physiological (electrical) signal can include several intracardiac electrograms (EGMs) sensed within a patient's heart and may include any number of features that may be ascertained by aspects of the system 50. Examples of cardiac physiological signal features include activation times, activations, activation waveforms, filtered activation waveforms, minimum voltage values, maximum voltages values, maximum negative time-derivatives of voltages, instantaneous potentials, voltage amplitudes, dominant frequencies, and peak-to-peak voltages. A cardiac physiological signal feature can refer to one or more features extracted from one or more cardiac physiological signals, derived from one or more features that are extracted from one or more cardiac physiological signals. Additionally, a representation, on a cardiac or a surface map, of a cardiac physiological signal feature may represent one or more cardiac physiological signal features, an interpolation of several cardiac physiological signal features. Each cardiac physiological signal also can be associated with a set of respective position coordinates that corresponds to the location at which the cardiac physiological signal was sensed. Each of the respective position coordinates for the sensed cardiac physiological signals can include three-dimensional Cartesian coordinates, polar coordinates, or another coordinate system. The cardiac physiological signals may be sensed on the cardiac surfaces, and the respective position coordinates can be on the endocardial surface, epicardial surface, in the mid-myocardium of the patient's heart, or in a vicinity.

During a signal-acquisition stage of a cardiac mapping procedure, the catheter 105 is displaced to multiple locations within the heart chamber into which the catheter 105 is inserted. At each location to which the catheter 105 is moved, the electrodes and sensors acquire physiological signals resulting from the electrical activity in the heart along with positional, or spatial, information of the catheter 105. The spatial information is used in building a three-dimensional grid of the anatomy during mapping. To perform a mapping procedure and reconstruct physiological information on the endocardium surface, the EAM system 70 may align a coordinate system of the catheter 105 with the endocardium surface's coordinate system, or vice versa. Alternatively, or additionally, the grid may be used to capture EGMs, and select mapping values based on statistical distributions associated with nodes of the grid. The EAM system 70 also can perform post-processing operations on the physiological information to extract and display useful features of the information to the operator of the system 50.

In generating an example electroanatomical map, a data stream including multiple signals, such as signals received from the mapping electrodes of the catheter 105, is input into the EAM system 70. During the automated electroanatomical mapping process, the data stream provides a collection of physiological and location signals that serve as an input to the mapping process. The signals may be collected directly by the mapping system, obtained from another system using an analog or digital interface, or both. The data stream can include signals such as unipolar and/or bipolar intracardiac EGMs, surface electrocardiograms (ECGs), electrode location information originating from one or more of a variety of methodologies, tissue proximity information, catheter force information, catheter to tissue contact information, catheter temperature, acoustic information, catheter electrical coupling information, catheter deployment shape information, electrode properties, respiration phase, blood pressure, and other physiological information. For the generation of specific types of maps, one or more signals may be used as one or more references to trigger and align the data stream relative to a cycle or clock, which can be used to create beat datasets. Beat metrics can be determined from the beat datasets. A beat acceptance process can be applied to determine which beat datasets will make up a map dataset. The map dataset may be stored in association with a three-dimensional grid that is dynamically generated during data acquisition.

Surface geometry data of the cardiac surface is generated, such as generated concurrently, during the data acquisition process using acceptance metrics employing a surface geometry construction process. This process constructs surface geometry using data such as electrode locations and catheter shape contained in the data stream. Additionally, or alternatively, previously collected surface geometry of the cardiac surface can be used as an input to surface geometry data. Previously collected geometry may have been collected using a different map dataset or using a different modality such as computerized tomography (CT), magnetic resonance imaging (MRI), ultrasound, or rotational angiography and registered to the catheter locating system. A surface map generation process is employed to generate surface map data from the map dataset and surface geometry data.

The depiction of the electrophysiology system 50 shown in FIG. 1 is intended for illustration or a general overview of the various components of the system 50 and is not intended to imply that the disclosure is limited to any set of components or arrangement of the components. For example, additional hardware components, such as breakout boxes or workstations, can be included in the electrophysiology system 50.

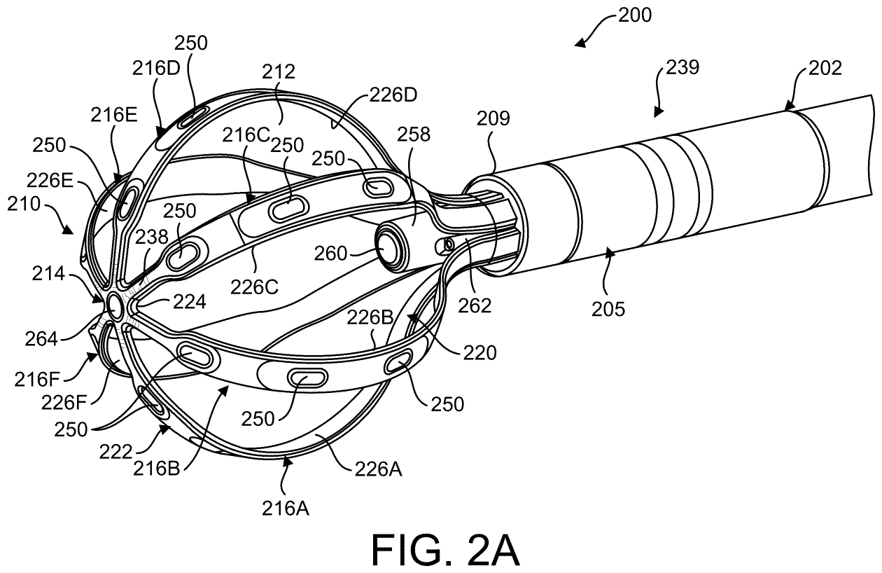

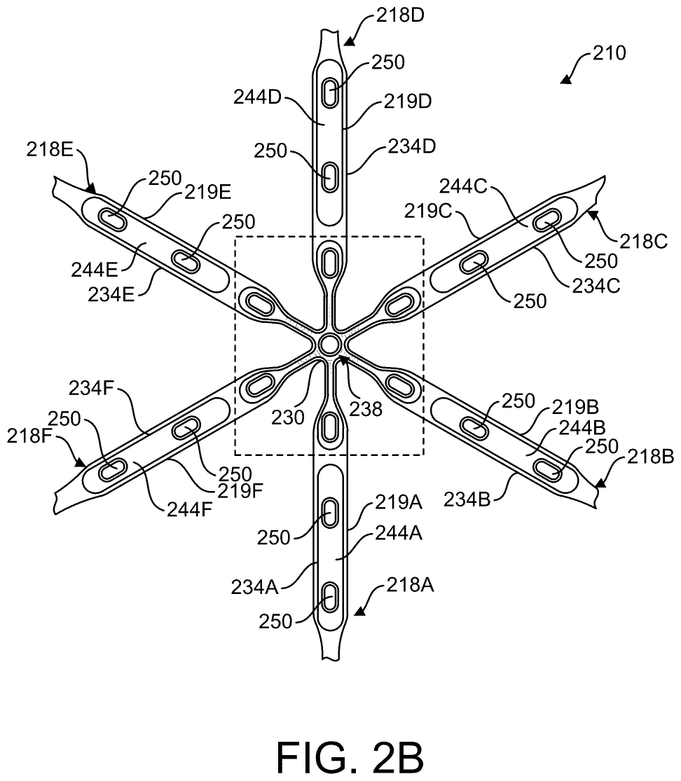

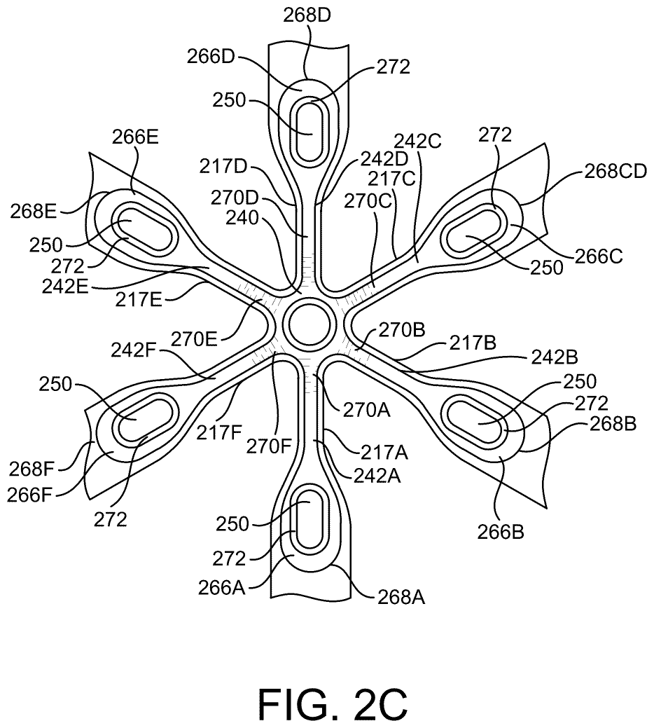

FIGS. 2A-2C illustrate an electroporation catheter 200 having a catheter distal portion 205 according to an embodiment of the present disclosure. The electroporation catheter 200 corresponds to the electroporation catheter 100 described with respect to FIG. 1. FIG. 2A illustrates the electroporation catheter 200 in an expanded configuration. The electroporation catheter 200 has a tubular outer shaft 208 having a shaft distal end 209, and an electrode assembly 210 extending distally from the distal end 209 of the outer shaft 208. In embodiments, the electrode assembly 210 is configured to self-expand from a collapsed configuration when constrained within a delivery sheath to a pre-defined expanded configuration defining an inner space 212. As will be explained in greater detail herein, the electrode assembly comprises one or more ablation electrodes configured to receive pulsed electrical signals/waveforms from the electroporation console 130 (FIG. 1), thereby creating pulsed electric fields sufficient for ablating target tissue via irreversible electroporation. Additionally, the electrode assembly 210 further includes a plurality of mapping and sensing electrodes configured for, among other things, sensing cardiac electrical signals, localization of the electrode assembly 210 within the patient anatomy (e.g., via the EAM system 70 of FIG. 1), and determining proximity to target tissue within the anatomy.

Overall, the electrode assembly 210 and other electrode assembly embodiments described herein within the scope of the present disclosure, is primarily designed for the creation of relatively localized ablation lesions (i.e., focal lesions), as compared to relatively large diameter circumferential lesions created in pulmonary vein isolation procedures). However, the skilled artisan will appreciate that the teachings of the present disclosure can be readily adapted for a catheter capable of large diameter circumferential lesions. The designs of the various electrode assembly embodiments described herein can provide the clinician with a wide range of capabilities for monopolar and bipolar focal pulsed field ablation of cardiac tissue, combined with the ability to perform localized (i.e., at the location of the delivery of pulsed field ablative energy), high fidelity sensing of cardiac tissue, e.g., for lesion or conduction block assessment, tissue contact determinations, and the like.

FIGS. 2B-2C are partial plan views the electrode assembly 210 of the electroporation catheter 200, shown in two-dimensions to illustrate the layout of the electrode assembly 210. Referring to FIGS. 2A-2C together, in the illustrated embodiment, the electrode assembly 210 as a whole has a distally-located central hub portion 214 and a plurality of splines 216A-216F extending proximally from the central hub portion 214. As further shown, each respective spline 216A-216F has a distal end portion 217A-217F, a proximal end portion 218A-218F, and an intermediate portion 219A-219F extending between the distal end portion 217A-217F and the proximal end portion 218A-218F. As shown, each of the proximal end portions 218A-218F is attached to and constrained by the distal end 209 of the outer shaft 202. As further shown, in the illustrated embodiment, the intermediate portion 219A-219F of each spline 216A-216F has a lateral width that is greater than the lateral width of each of the respective proximal end portion 218A-218F and the distal end portions 217A-217F. In embodiments, the particular geometry of the splines 216A-216F and the related components, e.g., ablation and mapping electrodes, is optimized to provide desired mechanical and therapeutic/diagnostic capabilities.

In the illustrated embodiment, the splines 216A-216F are composed of a support member 220 and a flexible circuit 222 secured to and disposed over an outer surface of the support member 222. The support member 220 functions, among other things, as a primary structural support of the electrode assembly 210, and thus primarily defines the mechanical characteristics of the electrode assembly 210. In embodiments, the support member 220 is formed from a superelastic material (metal or polymer) to provide desired mechanical/structural properties to the electrode assembly 210. In the case of the support member 220 formed of an electrically conductive superelastic material, the support member 220 includes a dielectric cover on the electrically conductive superelastic base material. In embodiments, the support member 220 is formed from an electrically conductive superelastic metal alloy, e.g., a nickel-titanium alloy. having a dielectric, or electrically insulative, cover, e.g., parylene or other suitable material.

The support member 220 includes a support member hub 224 and a plurality of support member branches 226A-226F. In embodiments, the support member branches 226A-226F are integrally formed with and extend proximally from the support member hub 224. For example, the entire support member 220 may be constructed from a single sheet of base material using conventional manufacturing techniques and an electrically insulative cover is applied to the base material. This unitary base material structure provides robust structural properties, for example, selective flexibility and enhanced fatigue characteristics, particularly in areas that are subject to relatively high stresses during manufacture and use of the electroporation catheter 200. Forming the support member 220 from a superelastic base material such as a nickel-titanium alloy (nitinol) facilitates configuring the support member 220 to assume its desired unconstrained shape such as shown in FIG. 2A due to the shape memory properties of the base material, while providing sufficient flexibility necessary to collapse the electrode assembly 210 within a delivery sheath. In embodiments, the support member branches 226A-226F can be selectively configured along their lengths to tune the mechanical characteristics of the electrode assembly 210.

The flexible circuit 222 includes a flex circuit hub 230 and a plurality of flex circuit branches 234A-234F. In embodiments, the flex circuit hub 230 is disposed over and secured to the support member hub 224. In embodiments, the flex circuit branches 234A-234F are integrally formed with the flex circuit hub 230, and each of the flex circuit branches 234A-234F is disposed over and secured to a respective one of the support member branches 226A-226F. The flexible circuit 222 comprises a layered construction including one or more dielectric substrate layers, and conductive traces formed thereon. Similar to the support member 220, the unitary construction of the flexible circuit 222 enhances its structural properties, for example, by minimizing joints or other discontinuities at regions subject to relatively high stresses during use.

As shown, the flexible circuit 222 includes a distal ablation electrode 238 that has a distal ablation electrode hub portion 240 and a plurality of radial segments 242A-242F. In the illustrated embodiment, the distal ablation electrode hub portion 240 is located on the flex circuit hub 230. Additionally, the radial segments 242A-242F are integrally formed with the distal ablation electrode hub portion 240. Each of the radial segments 242A-242F extends proximally along a portion of a respective one of the flex circuit branches 234A-234F. The flexible circuit 222 further includes a plurality of proximal ablation electrodes 244A-244F. As shown, each of the proximal ablation electrodes 244A-244F is located on a respective one of the flex circuit branches 234A-234F.

In some embodiments, the electrode assembly 210 includes an additional electrode 239, such as an additional electrode used for ablation, located on the shaft 208 near the distal end 209, such as to operate the catheter in a bipolar mode. For instance, the addition electrode 239, which can include a plurality of additional electrodes on the shaft 208, can be operated as one of the cathode or anode when ablation electrodes on the collapsible spline assembly 216A-216F are operated as the other of the anode or cathode such as for ablation. In the illustrated embodiment, the additional electrode 239 includes a pair of exposed ring electrodes around the outer portions of shaft 208. In other embodiments, the additional electrode 239 can be used in combination with other electrodes in the electrode assembly 210 to inject an electrical current.

As further shown, the flexible circuit 222 includes a plurality of spline sensing electrodes 250. In the illustrated embodiment, each of the spline sensing electrodes 250 is disposed within a periphery of one of the proximal ablation electrodes 244A-244F or one of the radial segments 242A-242F of the distal ablation electrode 238. For example, as shown, each of the distal-most spline sensing electrode 250 is disposed within a periphery of a respective one of the radial segments 242A-242F of the distal ablation electrode 238 and is electrically isolated from the distal ablation electrode 238. Additionally includes a plurality of the more proximally-located spline sensing electrodes 250 is disposed along and within a periphery of a respective one of each of the proximal ablation electrodes 244A-244F and electrically isolated therefrom.

In the particular illustrated embodiment, the electroporation catheter 200 includes a central post 258 extending distally from the distal end 209 of the outer shaft 202. As shown, the central post 258 extends partially into the inner space 212, and includes a post electrode 260. As further shown, in the particular illustrated embodiment, an optional irrigation lumen 261 is supported by the central post 258. In embodiments, the central post 258 may house additional components. For example, in embodiments, a magnetic navigation sensor (not shown) may be partially or wholly disposed within the central post 258. However, in other embodiments such a sensor may be located elsewhere on the electroporation catheter 200 (e.g., within the outer shaft 202). In the illustrated embodiment, the electrode assembly 210 further includes a hub sensing electrode 264 centrally located on the flex circuit hub 230.

The post electrode 260 can provide a number of functional advantages. In one example, the post electrode 260 can operate as a reference for unipolar electrograms, in lieu of reliance on surface ECG patch electrodes as are otherwise known in the art. The location of the post electrode 260, sometime referred to as a reference electrode in this context, for this purpose positions the reference much closer to the tissue being sensed than is possible with the conventional surface ECG approach, which may advantageously minimize far field noise and provide much sharper unipolar electrograms than what are possible using surface ECG electrodes. The post electrode 260 may also be operable to sense and measure other electrical parameters, e.g., voltages between it and the ablation electrodes or other sensing electrodes on the electrode assembly 210, thereby providing data usable for, in some examples, determining the shape of the electrode assembly during use (including when deformed by forces applied by cardiac walls), and displaying shape information via the EAM system 70 (FIG. 1).

In embodiments, the hub sensing electrode 264 allows tissue surface mapping to be conducted in a “forward” manner, eliminating the need to manipulate the electrode assembly 210 to place the spline sensing electrodes 250 against or proximate the tissue to be mapped. The inclusion of the hub sensing electrodes 264 further enhances bipolar sensing capabilities by providing for, in the illustrated embodiment, six additional bi-poles when paired with any of the distal-most spline sensing electrodes 250.

Referring in particular to FIG. 2C, each of the radial segments 242A-242F of the distal ablation electrode 238 includes proximal portion 266A-266F having a proximal end 268A-268F, and a distal portion 270A-270F extending from the distal ablation electrode hub portion 240. As further shown, a radial segment aperture 272 is formed in each proximal portion 266A-266F, and a respective one of the distal-most spline sensing electrodes 250 is disposed within each of the radial segment apertures 272. In the illustrated embodiment, each of the proximal portions 266A-266F has a greater lateral width than that of the corresponding distal portion 270A-270F, at least in part to accommodate the distal-most spline sensing electrodes 250.

The depiction of the catheter 200 in FIGS. 2A-2C is also intended for illustration or general overview of various components of a mapping and ablation catheter or catheter operated in an expanded configuration and is not intended to imply that this disclosure is limited to the particular arrangement of ablation electrodes or mapping electrodes or to the materials used to make circuit connections or the splines. For instance, the electrodes can be coupled to wires rather than flexible circuit traces and the electrode assembly can include more or fewer ablation electrodes or sensing electrodes.

The electrophysiology system 50 is capable of detecting, or are configured to detect, electrical characteristics, such as impedance, which can correspond with several properties including myocardial tissue proximity to an electrode. For example, the system 50 utilizes impedance measurements to sense contact between an electrode on the catheter 105, such as catheter 200, and tissue. In general, the impedance of a given medium is determined based upon applying a known voltage or current to a given medium and measuring the resulting current or voltage. In some embodiments, impedance measurements of a given medium can be obtained by injecting current between two electrodes and measuring the resulting voltage between the same electrodes through which the current was injected. In one example, the controller, such as controller of the electroporation console 130 or the mapping and navigation controller 90, can select and inject a current between any two electrodes on the catheter 200 and measure a resulting voltage between the same electrodes. In this example, impedance is determined in a two-terminal configuration. The ratio of the voltage potential to the applied current provides an indication of the impedance of the medium through which the current traveled, which can be determined via the controller.

For example, a current can be injected between distal ablation electrode 238 and a shaft ring electrode 239. Impedance of the medium (e.g., cardiac tissue) adjacent to distal ablation electrode 238 and shaft ring electrode 239 can be measured according to the methodology disclosed above. For example, if the current is injected between electrodes embedded in cardiac tissue, the impedance of the cardiac tissue may be determined.

In some instances, system 50 may utilize different impedance measurements of a local medium to determine whether the distal ablation electrode 238 is contacting tissue. For example, the impedance of cardiac tissue is different than that of the impedance of blood. Therefore, by knowing the relative difference in the impedances of tissue versus blood, system 50 may be able to determine whether the medium through which a current is being applied is either blood or cardiac tissue, for example.

In some embodiments, impedance can be determined in a four-terminal configuration. In general, the four-terminal configuration drives current through a pair of electrodes of the electrode array 210 and measures voltage across a different pair of electrodes. For example, current is injected via a current carrying pair of electrodes and voltage is measured across a sensing pair of electrodes. In one advantageous application, a four-terminal configuration may not be sensitive to the impedance of the electrodes themselves. For instance, the measured impedance in a two-terminal configuration includes the surrounding medium and both electrodes. In contrast, the four-terminal configuration measures the voltage across the sensing pair of electrodes in which the current is negligible. Consequently, the measured impedance is that of the surrounding medium and is largely independent of the impedance of the sensing pair of electrodes and their interface with the surrounding medium.

In one example, a current can be injected between the distal ablation electrode 238 and a shaft ring electrode 239, which define the current carrying pair of electrodes. A pair of distal-most spline sensing electrodes 250 on adjacent spline such as splines 216B, 216C can define the sensing electrode pair. The current is injected between the distal ablation electrode 238 and the shaft ring electrode 239, and voltage is measured across the distal-most spline sensing electrodes 250 on adjacent splines 216B, 216C. The impedance measurement is the impedance of the medium between the sensing pair of electrodes, or the medium adjacent the distal-most spline sensing electrodes 250 on adjacent splines 216B, 216C in this example. For instance, if the distal-most spline sensing electrodes 250 on adjacent splines 216B, 216C are abutted against cardiac tissue, the distal-most spline sensing electrodes 250 on adjacent splines 216B, 216C measure the impedance of the cardiac tissue.

In some embodiments, any pair of electrodes of the electrode assembly 210 can implemented as current carrying pair of electrodes from which current is injected, and any pair of electrodes of the electrode assembly 210 can be implemented as the sensing pair of electrodes across which voltage is measure. In some embodiments, the four-terminal configuration is implemented via three electrodes, such as one of the electrodes (a dual-purpose electrode in the configuration) is included in the current carrying pair of electrodes and the sensing pair of electrodes. This configuration can approximate the four-terminal configuration if the impedance of the dual-purpose electrode is low and not expected to vary significantly. Additionally, one or more of the electrodes in a four-terminal configuration can be located off the catheter 200, such as on a pad coupled to the patient's skin or on another surgical device.

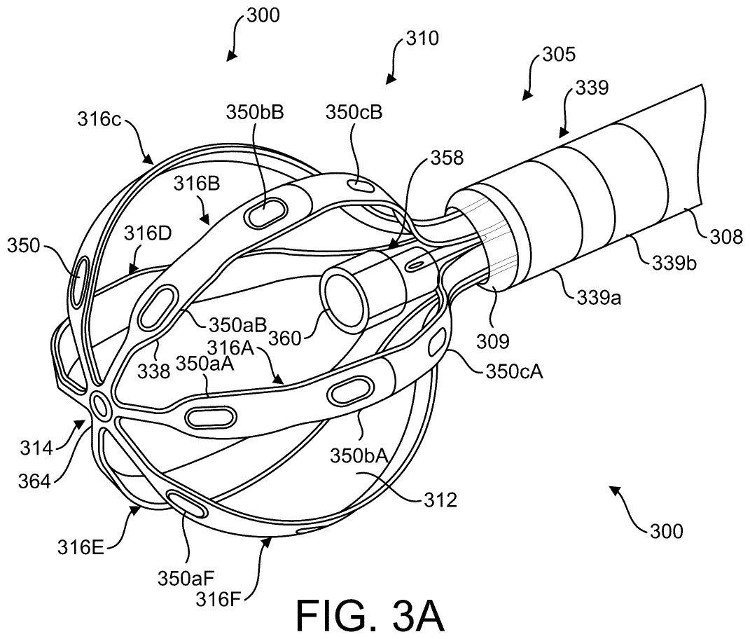

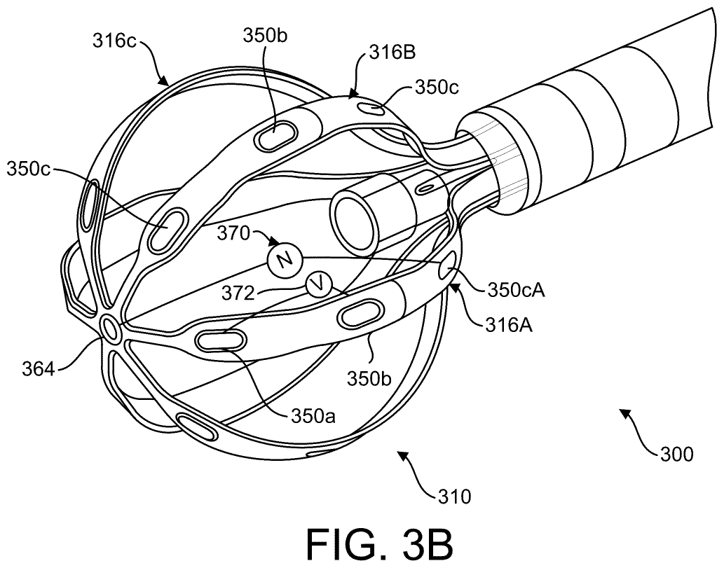

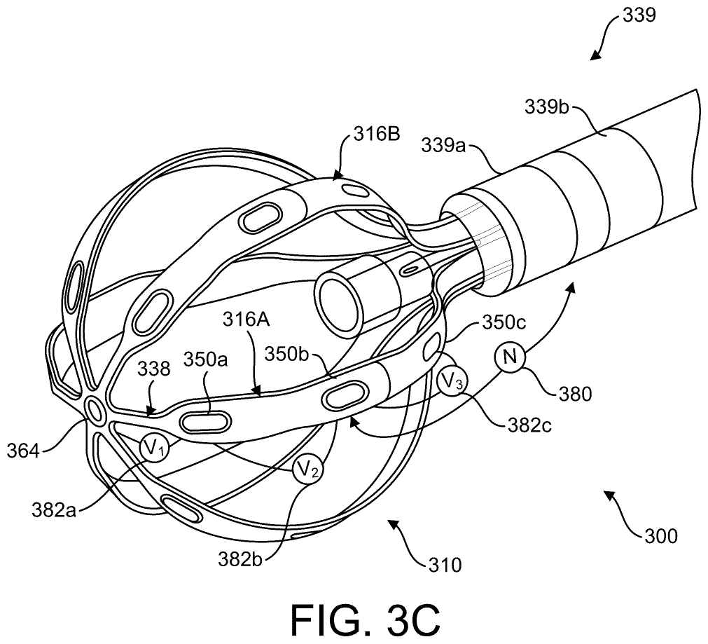

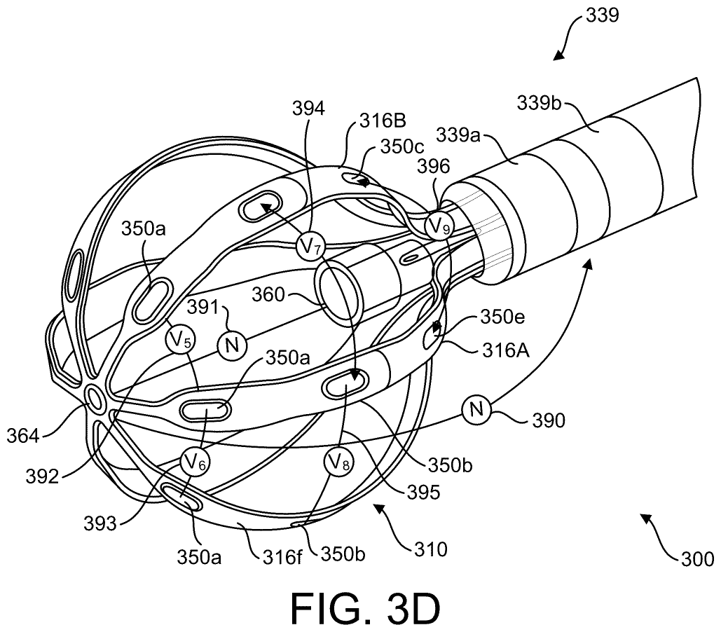

FIG. 3A illustrates another self-expanding, generally spherical electroporation catheter 300 having multiple ablation and sensing electrodes that can be configured via a controller to detect electrical characteristics such as impedance and corresponds with the electroporation catheter 100 described with respect to FIG. 1. The electroporation catheter 300 includes a catheter distal portion 305 and has a tubular outer shaft 308 having a shaft distal end 309, and an electrode assembly 310 extending distally from the distal end 309 of the outer shaft 308. In embodiments, the electrode assembly 310 is configured to self-expand from a collapsed configuration when constrained within a delivery sheath to a pre-defined expanded configuration defining an inner space 312. The electrode assembly 310 comprises one or more ablation electrodes configured to receive pulsed electrical signals/waveforms from the electroporation console 130 (FIG. 1), thereby creating pulsed electric fields sufficient for ablating target tissue via irreversible electroporation. Additionally, the electrode assembly 310 further includes a plurality of mapping and sensing electrodes configured for, among other things, sensing cardiac electrical signals, localization of the electrode assembly 310 within the patient anatomy (e.g., via the EAM system 70 of FIG. 1), and determining proximity to target tissue within the anatomy.

The electrode assembly 310 has a distally-located central hub portion 314 and a plurality of splines 316A-316F extending proximally from the central hub portion 314. Each respective spline 316A-316F has a distal end portion, a proximal end portion, and an intermediate portion extending between the distal end portion and the proximal end portion similar to that of catheter 200. Each of the proximal end portions is attached to and constrained by the distal end 309 of the outer shaft 302. In one illustrated embodiment, the splines 316A-316F are composed of a support member and a flexible circuit secured to and disposed over an outer surface of the support member similar to catheter 200.

Electrodes of the electrode assembly 310 can be constructed similarly to the electrodes of catheter 200 and electrically coupled to a catheter proximal portion as in catheter 200. As shown, the electrode assembly 310 includes a distal ablation electrode 338 as a pad that has a distal ablation electrode hub portion and a plurality of radial segments. The electrode assembly 310 includes an additional electrode 339, such as a plurality of additional or shaft electrodes 339a, 339b as ring electrodes, that can be used in combination with ablation electrode 338 in bipolar mode, located on the shaft 308 near the distal end 309, such as to operate the catheter in a bipolar mode. As shown, a post electrode 360 (or reference electrode) is included as a pad electrode on a central post 358 that extends partially into the inner space 312. Further, the electrode assembly 310 includes a plurality of spline sensing electrodes 350 as pads on each spline 316A-316F. In the illustrated embodiment, each spline 316A-316F includes a distal spline sensing electrode 350a, such as illustrated distal spline sensing electrodes 350aA, 350aB; intermediate spline sensing electrode 350b, such as illustrated intermediate spline sensing electrodes 350bA, 350bB; and a proximal spline sensing electrode 350c, such as illustrated proximal spline sensing electrodes 350cA, 350cB. In the illustrated example, spline sensing electrodes 350a, 350b are disposed within a periphery of the distal ablation electrode 338. Still further, the electrode assembly 310 includes a hub sensing electrode 364 disposed on the central hub portion 314 within the periphery of the distal ablation electrode 338.

FIG. 3B illustrates an example four terminal configuration to determine local impedances of the electrode assembly 310 on catheter 300. Local impedance is measured on each spline 31A-316F using the hub sensing electrode 364 and the proximal spline sensing electrode 350c on one spline as a current carrying pair of electrodes to drive current 370, such as on spline 316A. The distal spline sensing electrode 350a and the adjacent intermediate sensing electrode 350b on the spline, such as spline 316A, are the sensing pair of electrodes used to measure voltage 372. In one embodiment, the impedance is determined for each spline in this manner. For example, the impedance for spline 316A is measured by injecting current via current carrying pair of electrodes 364, 350c on spline 316A, and measuring the voltage across sensing pair of electrodes 350a, 350b on spline 316A; the impedance for spline 316B, is measured by injecting current via current carrying pair of electrodes 364, 350c on spline 316B, and measuring the voltage across sensing pair of electrodes 350a, 350b on spline 316B; the impedance for spline 316C is measured by injecting current via current carrying pair of electrodes 364, 350c on spline 316C, and measuring the voltage across sensing pair of electrodes 350a, 350b on spline 316C, and so forth for each spline of the plurality of splines 316A-316F. Because each spline employs the hub sensing electrode 364 in the circuit of the four terminal configuration to determine local impedance, impedance measurements are multiplexed in time or frequency and measured for each spline frequently enough to account for movement of the catheter 300. For example, the impedance for spline 316A is measured prior to the measurement of impedance for spline 316B, which is measured prior to the measurement of impedance for spline 316C, and so on.

FIG. 3C illustrates another example four terminal configuration to determine local impedances of the electrode assembly 310 on catheter 300. Local impedance is measured on each spline 31A-316F using the ablation electrode 338 and one or both of the additional or shaft electrodes 339a, 339b as a current carrying pair of electrodes to drive current 380. The hub electrode 364 and distal spline sensing electrode 350a on a spline 316A comprise a first sensing pair of electrodes to measure a first voltage v₁ 382a, the distal spline sensing electrode 350a and adjacent intermediate spline sensing electrode 350b on spline 316A comprise a second sensing pair of electrodes to measure a second voltage v₂ 382b, and the intermediate spline sensing electrode 350b and adjacent proximal spline sensing electrode 350c on spline 316A comprise a third sensing pair of electrodes to measure a third voltage v₃ 382c. In one embodiment, the impedance is determined along each spline for each spline in this manner. For example, the impedances along spline 316A are measured by injecting current via current carrying pair of electrodes 338, 339, and measuring the first voltage across first sensing pair of electrodes 364, 350a, measuring the second voltage across second sensing pair of electrodes 350a, 350b, and measuring the third voltage across third sensing pair of electrodes 350b, 350c all on spline 316A; the impedances along spline 316B are measured by injecting current via current carrying pair of electrodes 338, 339, and measuring the first voltage across first sensing pair of electrodes 364, 350a, measuring the second voltage across second sensing pair of electrodes 350a, 350b, and measuring the third voltage across third sensing pair of electrodes 350b, 350c all on spline 316B; and so on. In one embodiment, impedance measurements along a spline are multiplexed in time or frequency and measured for each sensing pair of electrodes on a spline, and impedance measurements are multiplexed in time or frequency and measured for each spline frequently enough to account for movement of the catheter 300. For example, the impedances along spline 316A are measured prior to the measurement of impedance along spline 316B, which is measured prior to the measurement of impedances along spline 316C, and so on.

FIG. 3D illustrates still another example four terminal configuration to determine local impedances of the electrode assembly 310 on catheter 300. Local impedance can be measured for each spline sensing electrode 350 using the hub sensing electrode 364 and one or both of the additional electrodes 339a, 339b as a current carrying pair of electrodes to drive a current 390 or the hub sensing electrode 362 and the post electrode 360 as a current carrying paid of electrodes to drive a current 391. Any two sensing electrodes 350, such as distal sensing electrodes 350a on adjacent splines or adjacent sensing electrodes on the same spline, can comprise a sensing pair of electrodes to measure a voltage v. In the illustrated example, distal sensing electrodes 350a on adjacent splines 316A and 316B measure voltage v₅ 392, distal sensing electrodes 350a on adjacent splines 316A and 316F measure voltage v₆ 393, intermediate sensing electrodes 350b on adjacent splines 316A and 316B measure voltage v₇ 394, intermediate sensing electrodes 350b on adjacent splines 316A and 316F measure voltage v₈ 395, proximal sensing electrodes 350c on adjacent splines 316A and 316B measure voltage v₉ 392, and so on. In some examples, the hub electrode 364 can be included in the current carrying pair of electrodes and the sensing pair of electrodes as a dual-purpose electrode in the configuration. Different frequencies of the injected current can be applied to determine a preferred frequency for use in the impedance measurement based on different states of cardiac tissue. In one embodiment, impedance measurements between sensing pairs of electrodes on adjacent spline are multiplexed in time or frequency and measured for each adjacent sensing pair of electrodes on an adjacent spline, and impedance measurements are multiplexed in time or frequency and measured for each spline frequently enough to account for movement of the catheter 300.

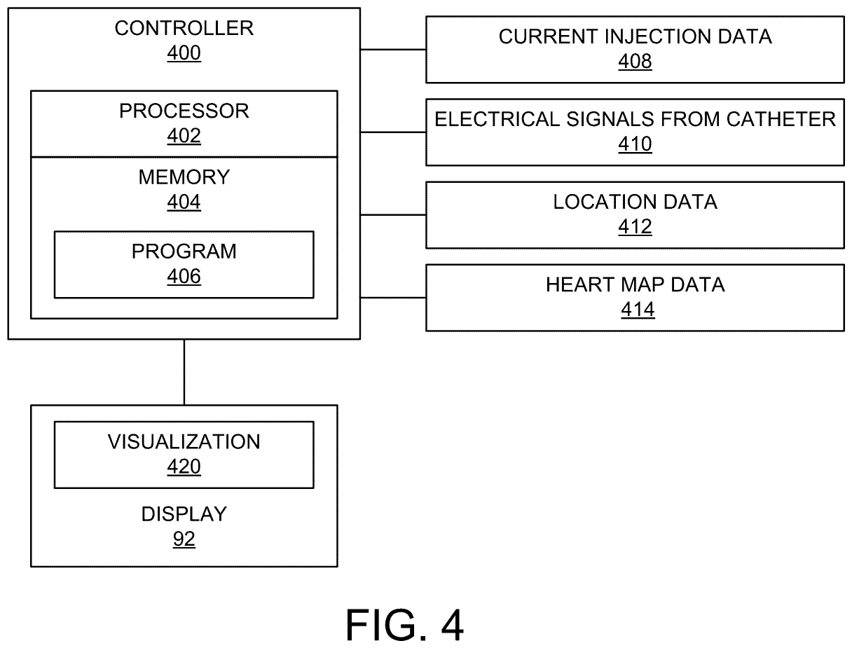

FIG. 4 illustrates an example controller 400 that can be used with the example electrophysiology system 50, such as a controller of the example electroporation catheter system 60, which may include a controller of the electroporation console 130, or a controller of the example EAM system 70, which may include a mapping and navigation controller 90. The controller 400 can include a processor 402 and a memory 404. The memory 404 stores processor executable instructions 406. In one example, the processor executable instructions 406 can be in the form of a program, such as a computer program or application. In one example, the controller 400 can be implemented to include a computing device such as a laptop computer, a workstation, a desktop computer, or a tablet. The controller 400 can be implemented to include or be coupled to additional components such as a graphical display, a touchscreen, speakers or other output devices, a keyboard or other input devices, or communication circuitry such as computer network adapters. In one example, the controller 400 is coupled to display 92. The controller 400 may be implemented in a variety of architectures and components, such as the processor 402 and memory 404, may be distributed in various locations. In some embodiments, the processor 402 includes one or more main processing cores to run an operating system and perform tasks on an integrated circuit, and the processor 402 can also include built-in logic or a programmable functional unit, also on the same integrated circuit. Memory 404 is an example of computer storage media. Computer storage media includes RAM, ROM, EEPROM, flash memory or other memory technology, CD-ROM, digital versatile discs (DVD) or other optical storage, magnetic cassettes, magnetic tape, magnetic disk storage or other magnetic storage devices, USB flash drive, flash memory card, or other flash storage devices, or other storage medium that can be used to store the desired information and that can be accessed by the processor 402. Any such computer storage media may be part of the controller 400 and implemented as memory 404. Memory 404 is a non-transitory, processor readable memory device. Accordingly, a propagating signal by itself does not qualify as storage media or memory 404.

The controller 400 is configured to receive inputs to and generate outputs from the electrophysiology system 50. For example, the controller 400 can generate outputs such as current injection data 408 to the console 130 to generate currents in selected electrodes of the catheter 105 to measure impedance. Also, the controller 400 can receive electrical signals 410 from the catheter 105, such as catheters 200 and 300. For example, electrical signals 410 can be received from electrodes on the electrode array in response to signals generated via the catheter, such as injected currents or pulsed field ablation or from physiological signals such as EGMs. Additionally, the controller 400 can receive location data 412 such as information regarding the location of the electrode array from the EAM system 70. In some embodiments, the controller 400 can receive an input representative of the anatomical map of the heart, or heart map data 414, which heart map data 414 include the data regarding representations of the geometric anatomical map of the heart and the electro-anatomical map of the heart, such as from the EAM system 70. Further, the controller 400 generates a visualization 420, such as transmits to a graphic display a signal for a visualization of the measured impedance on the catheter 105.

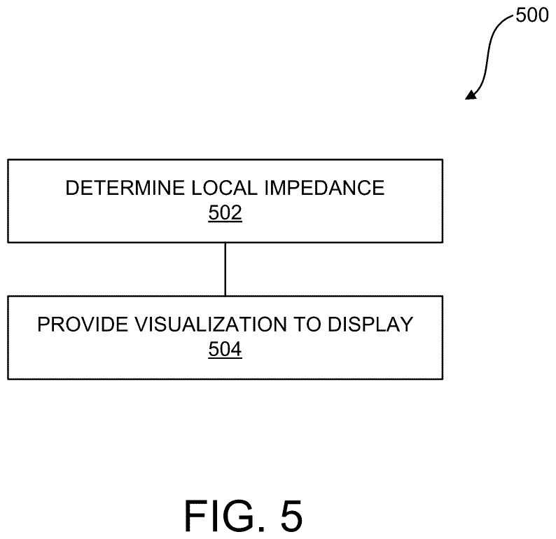

FIG. 5 illustrates a process 500 to configure a controller, such as controller 400. In one embodiment, process 500 can be implemented as set of processor-executable instructions, such as instructions 406, stored in a non-transitory memory, such as memory 404 to be executed by a processor 402 to configure controller 400. The instructions to implement process 400 can be configured to receive information, such as to retrieve from memory 404, data regarding electrical signals 410, location data 412, and heart map data 414. Further, the instructions to implement process 400 can be configured to perform impedance measurements, annotate, adjust, or write to heart map data 414 and to generate a visualization, such as visualization 420 to transmit to a display such as display 92. The graphical representation can include a schematic representation or other indicia that presents impedance of a model of electrode assembly or impedances of on a plurality of locations of a model of the electrode assembly, and, in some embodiments, the location of the electrode assembly of the electroporation catheter 105 with respect to the heart such as may be determined from the location data 412. Impedances can change as the catheter moves or the anatomy moves, and the graphical representation of the impedances can be changed and updated as well, such as quickly enough to appear as real time. In one example, measured impedances can be presented on the graphical display as colors depicting a range of impedances or as vectors depicting the measured impedance with respect to locations on the electrode assembly.

Local impedance is determined from a four-terminal configuration of the electrode array at 502. For example, the controller 400 selects a current carrying pair of electrodes on the electrode array. In some embodiments, the controller selects a current amplitude and frequency of the injected current to the current carrying pair of electrodes. In some embodiments, the controller 400 provides an output to the direct the console 130 to inject the current to the selected current carrying pair of electrodes, and the current is injected via the console 130 to the selected current carrying pair of electrodes. The controller 400 selects the sensing pair of electrodes to measure voltage. In some embodiments, controller 400 provides an output to the direct the console 130 to receive electrical signals from the selected sensing pair of electrodes, and the voltage is measured via the selected sensing pair of electrodes and, in some embodiments, provided to the controller 400. In some embodiments, the controller cycles through several sets of selected sensing pairs of electrodes during an injection of current, such as selected sensing pairs of electrodes are multiplexed in time or frequency, to determine several voltage measurements. In one embodiment, the selected sensing pairs of electrodes are multiplexed in time or frequency while the electrode array is at a relatively stationary location with respect to the heart.

In some embodiments, the impedance determinations at 502 are performed either concurrently or sequentially with other functions of the electrode array, such as ablation or other data collection in a known manner.

In some embodiments, the impedance of a spline of the plurality of splines is determined via the four-terminal configuration at 502. Examples of current carrying pairs of electrodes are illustrated in FIGS. 3B-3D. The controller 400 can cycle through various sets of sensing pairs of electrodes as illustrated in FIGS. 3B-3D to determine the local impedances of each spline. Current is injected to a current carrying pair of the plurality of electrodes and a voltage is measured across a sensing pair of electrodes comprising a sensing electrode of the plurality of sensing electrodes located on that spline. In one example of the four-terminal configuration, the current carrying pair of electrodes can include hub sensing electrode 364 and the proximal spline sensing electrode 350c on one spline of electrode assembly 300, and the sensing pair of electrodes can include the distal spline sensing electrode 350a and the adjacent intermediate sensing electrode 350b on the spline. Impedance is determined for each spline via cycling through a current carrying pair of electrodes and a sensing pair of electrodes on each spline. In another example of the four-terminal configuration, the current carrying pair of electrodes include the ablation electrode 338 and one or both of the additional electrodes 339a, 339b. The hub electrode 364 and distal spline sensing electrode 350a on a spline 316A comprise a first sensing pair of electrodes to measure a first voltage v₁ 382a, the distal spline sensing electrode 350a and adjacent intermediate spline sensing electrode 350b on spline 316A comprise a second sensing pair of electrodes to measure a second voltage v₂ 382b, and the intermediate spline sensing electrode 350b and adjacent proximal spline sensing electrode 350c on spline 316A comprise a third sensing pair of electrodes to measure a third voltage v₃ 382c. In one embodiment, all of the voltage measurements are taken simultaneously on a spline, and the voltage measurements for each spline are taken one at a time. In another embodiment, all of the voltage measurements are taken simultaneously on the plurality of splines. In still another embodiment, local impedance can be measured for each spline sensing electrode 350 using the hub sensing electrode 364 and one or both of the additional electrodes 339a, 339b as a current carrying pair of electrodes to drive a current 390 or the hub sensing electrode 362 and the post electrode 359 as a current carrying paid of electrodes to drive a current 391. Any two sensing electrodes 350, such as distal sensing electrodes 350a on adjacent splines or adjacent sensing electrodes on the same spline, can comprise a sensing pair of electrodes to measure a voltage v.

From the voltage measurement of a selected sensing pair of electrodes based on the injected current from a selected current carrying pair of electrodes, the controller 400 can determine local impedance in a known manner. The ratio of the measured voltage to the injected current provides an indication of the local impedance, such as the impedance of the medium, through which the current traveled. The ability of the controller 400 to accurately measure impedance may depend on the relative distribution of the current density being applied to a medium. The larger spacing between the hub sensing electrode 364 or ablation electrode 338 and the shaft electrodes 339 or post electrode 359 provides advantages in injecting current. The larger amount, frequent spacing on the splines and electrode array, and relatively smaller size of the sensing electrodes 350 provides advantages in using the sensing electrodes 350 to measure voltages.

The local impedance obtained from the four-terminal configuration is provided as a graphical representation of a model and transmitted to the display 92 as a visualization at 504. In one embodiment, the controller 400 generates a graphical representation of the electrode array or distal end of the catheter and models the local impedance determined from the four-terminal configuration on the graphical representation. The graphical representation can allow a clinician to readily determine whether to apply therapy in a selected location. For example, graphical representations of local impedance measurements can provide the benefits of assessing tissue contact and provide information about which parts of the electrode array are in contact with tissue. Also, local impedance information can be useful in discriminating different tissue types and assessing the success of pulse field ablation. In one embodiment, the visualization can be generated in a mapping system included with EAM system 70 or via a software widget on a map.

In another embodiment of the local impedance obtained from the four-terminal configuration is provided as a graphical representation of a model and transmitted to the display 92 as a visualization at 504, the visualization is an enhanced anatomical or electro-anatomical map of the heart. In one embodiment, local impedance data can be applied to distinguish media on the map of the heart, such as to distinguish scar tissue from other myocardial tissue and from blood because scar tissue includes a different impedance signature than other heart tissue and blood that may not be discernible from other processes to map the heart structures. The local impedance data generated at 502 can be applied to a heart map to produce maps of scars and correlate electrical information, such as voltage or activation with other heart map data 414, such as CT or MRI image data of the heart for further investigation during a heart procedure.

In another example, local impedance information can be combined with other data to increase specificity and sensitivity of information. For example, impedance measurements may not have a strong enough specificity for tissue contact such as relative to a blood pool due to differing characteristics of tissue impedance. The local impedance information could be combined with information or data received from other systems to enhance sensitivity for tissue contact. For instance, local impedance information can be combined with signal strength or slew rate of unipolar electrograms obtained from sensing or mapping relative to indifferent, reference, or probe electrodes within the tip, or strength or slew rate of bipolar electrograms between sensing electrodes on the tip. A uniform impedance across all electrodes, for example, on a catheter can indicate the catheter is disposed in a blood pool, and any changes in the impedance on a cluster of electrodes can indicate contact with tissue on the electrodes—and thus differential impedance over time can indicate tissue contact even if the local impedance is lower or different than expected for healthy tissue.

In another example of generating an enhanced map of the heart, the local impedance can be applied to resolve structures of the models of the heart. For example, the mapping features of the catheter 105 can be used in combination with the EAM system 70 to generate high fidelity three-dimensional anatomical and electro-anatomical maps of the heart, including portions of the heart such as cardiac chambers of interest or other structures of interest such as the sinoatrial node or atrioventricular node. The anatomical and electroanatomical maps are generated for presentation on display 92. In the embodiment, local impedance information obtained from the electrode assembly can be applied to resolve difficult to obtain concave or interior structures such as moderator band via contact with the electrode assembly. Local impedance information can be applied to enhance the shape and location of structures. For example, the geometry of the map can be trimmed or enhanced based on locations where tissue contacts the electrode array.

In still another example of generating an enhanced map of the heart, the local impedance can be applied to limit data collection to tissue areas in contact with the electrode assembly. For instance, electrical data or geometry received by the electrode assembly from areas of tissue not in contact with the electrode assembly could be flagged for review or editing. In one embodiment, the local impedance determined at 502 can be correlated with data collected via other data gathering functions, such as mapping. For instance, the system can configure the catheter 105 to collect data for mapping and determine local impedance data at the same time. Data collected for mapping, such as EGMs, can be received via electrodes in contact with tissue and via electrodes not in contact with tissue, such as far-field EGMs, as determined via local impedance data of the electrode receiving the EGM. EGMs or other electro-anatomical data collected from electrodes not in contact with tissue can be assigned weights that are less than EGMs or other electro-anatomical data collected from electrodes in contact with tissues to generate enhanced heart maps. In another embodiment, EGMs or other electro-anatomical data is collected if local impedance is above a selected threshold. In one instance, electro-anatomical data is collected on a beat-by-beat basis with respect to tissue contact information as determined via local impedance information at 502.

In still another example, the local impedance information can be combined with functionality of the electroporation console 130 or generator. In one example, the local impedance information can be applied to the generator to permit activation of the generator and allow an ablation to proceed if the local impedance information is within a certain range to indicate tissue contact. Also, the local impedance information can be applied to the generator to block activation of the generator and not allow an ablation to proceed if the local impedance information is with a certain range to indicate poor contact or in a blood pool—or without a range to indicated strong tissue contact. This can prevent wasted ablation attempts.

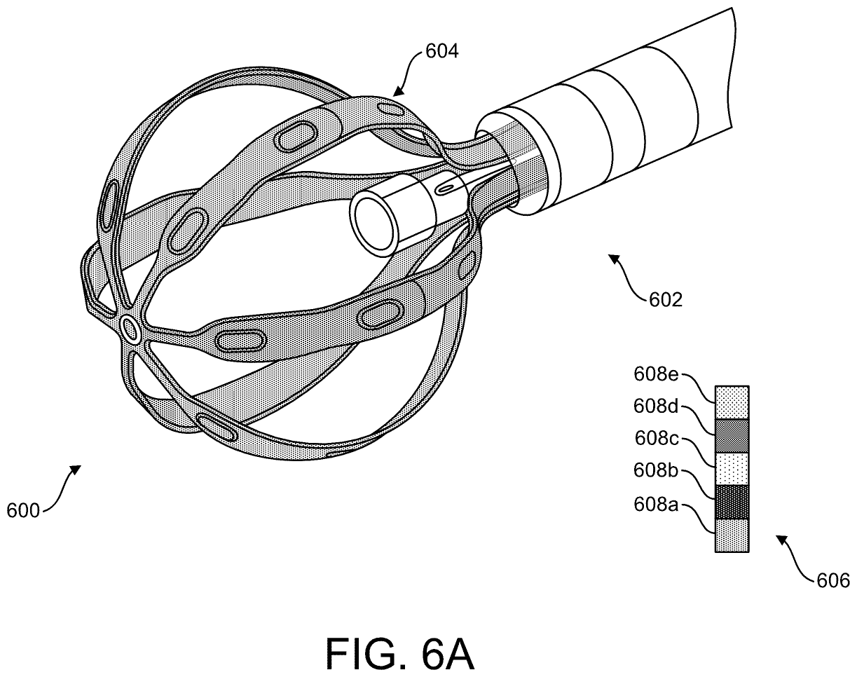

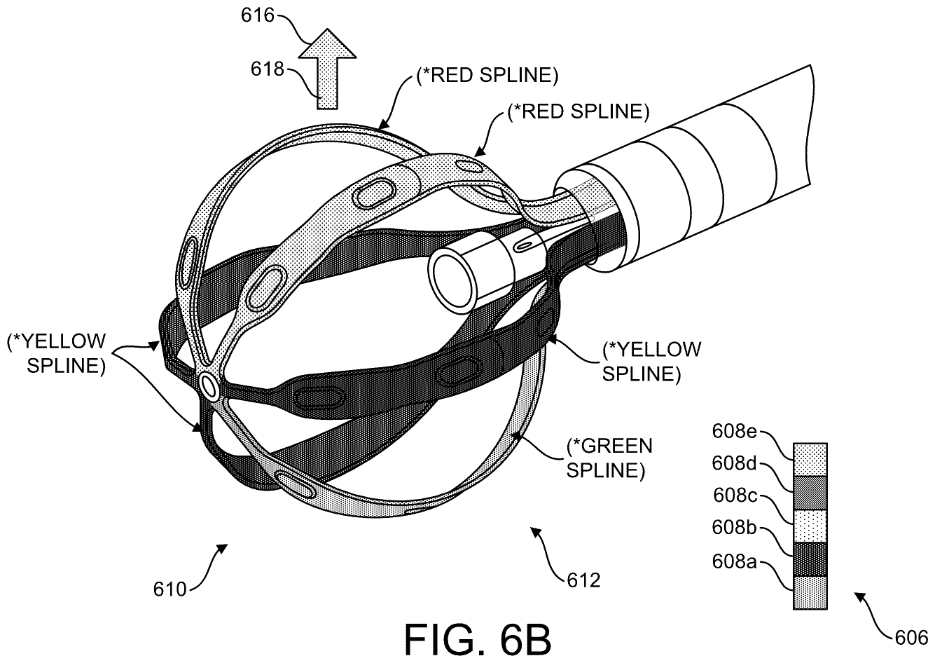

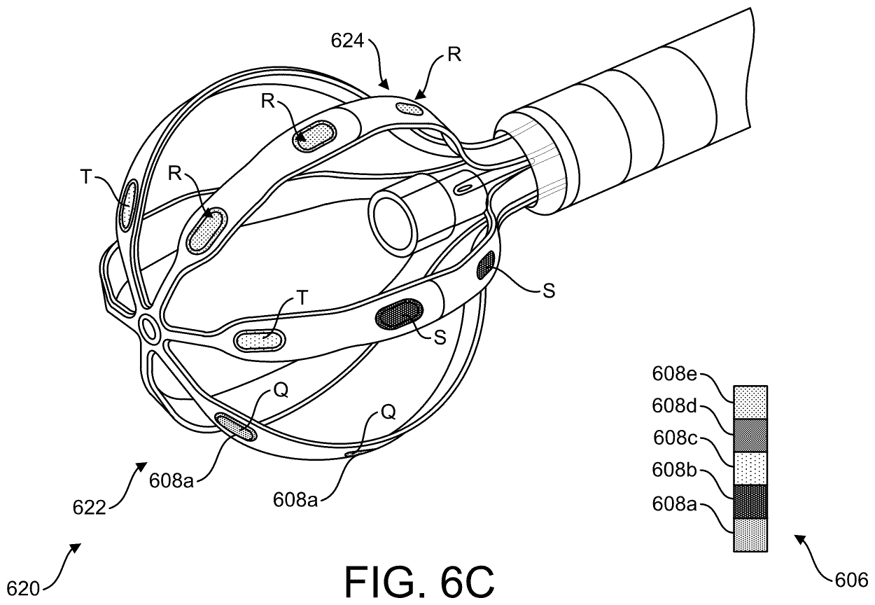

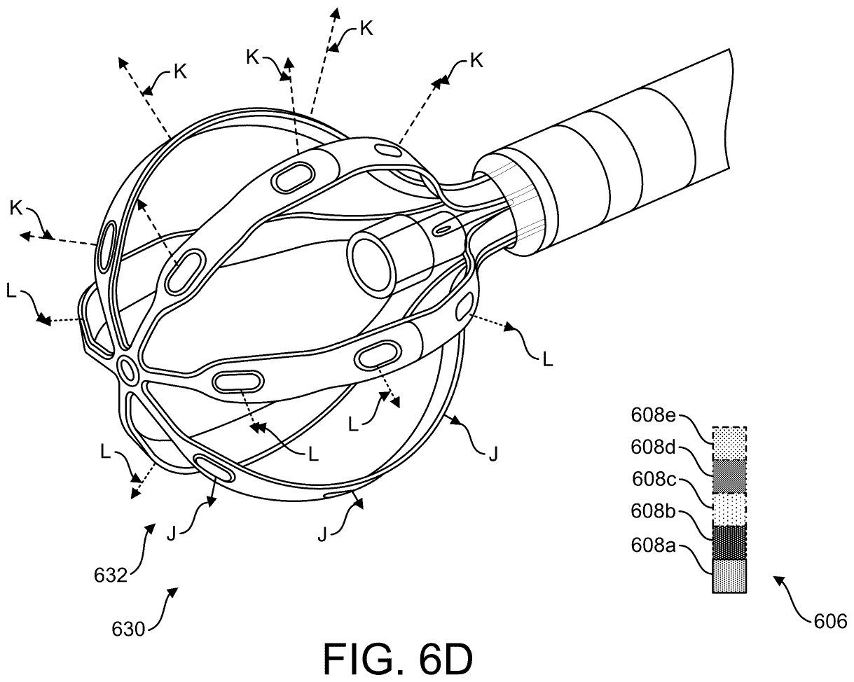

FIGS. 6A-6D illustrate example visualizations including graphical representations of a catheter, such as catheter 300, modeling local impedance on the catheter. The example visualizations apply color indicators or vectors to present impedance measurements. Visualizations can also be annotated or indicated with measurements of impedance or labels of mediums corresponding with the impedance such as blood and types of cardiac tissue.

FIG. 6A illustrates an example visualization 600 including a catheter model 602 of the self-expanding electroporation catheter 300. Impedances have been determined according to process 500 from the four-terminal configuration at 502. In the illustrated example, the measured impedances at 502 have been processed to obtain an average impedance of the electrode assembly 310. The average impedance of the electrode assembly 310 is graphically represented on the catheter model 602 of the visualization as a color 604 representative of the impedance measurement selected from a color scale 606 of multiple ranges of impedances as provided at 504. In one embodiment, the color scale 606 is based on the range of impedances that may be encountered within the cardiac anatomy. For example, the color scale can include a plurality of colors or a plurality of shades of colors corresponding with ranges of impedances from a selected low impedance value to a selected high impedance value. In another embodiment, the color scale is based on the media that may be encountered within the cardiac anatomy. For example, the color scale can include a plurality of colors or a plurality of shades of colors corresponding with different media, such as a color corresponding with blood and a color corresponding with myocardial tissue.

In the illustrated example, the color scale 606 includes five different colors 608a, 608b, 608c, 608d, 608c, in which each color corresponds with a range of impedances from a selected low impedance to a selected high impedance. In the illustrated example, first color 608a corresponds with a low range of impedances and can be represented by a color on a color display such as green. The second color 608b corresponds with a second lowest range of impedances and can be represented by a color on the color display such as yellow. The third color 608c corresponds with a middle range of impedances and can be represented by a color on the color display such as orange. The fourth color 608d corresponds with a second highest range of impedances and can be represented by a color on the color display such as pink. The fifth color 608e corresponds with a highest range of impedances and be represented by a color on the color display such as red.

In the illustrated example of visualization 600, in which the electrode array is graphically represented on the catheter model 602 of the visualization as a single color 604 representative of the impedance measurement selected from a color scale 606 of multiple ranges of impedances, the catheter model 602 is graphically represented as the first color 608a, in which the average impedance of the electrode array is within the lowest range of impedances of the color scale.

FIG. 6B is another example visualization 610 including a catheter model 612 of the self-expanding electroporation catheter 300. Impedances have been determined according to process 500 from the four-terminal configuration at 502. In the illustrated example, the measured impedances at 502 have been processed to obtain impedance values for each spline, or average impedance values for each spline, of the electrode assembly 310. The impedance values for each spline of the electrode assembly 310 are graphically represented on the catheter model 602 of the visualization as colors 614 representative of the impedance measurement selected from the color scale 606 (in FIG. 6A) of multiple ranges of impedances as provided at 504.

In the illustrated example visualization 610, the splines of the catheter model 612 are represented with different colors from the color scale 606 based on the determined impedance values of the splines. For instance, the first spline A of catheter model 612 is presented in the first color 608a indicating that the local impedance value of the corresponding spline on catheter 300 is within the lowest range of impedances on the scale 606. For example, the spline corresponding with the first spline A may be in a blood pool. Splines C and D of catheter model 612 are presented in the fifth color 608e indicating that the local impedance value of the corresponding spline on catheter 300 is within the highest range of impedance on the scale 606. For example, the splines corresponding with splines C and D may be in direct contact with cardiac tissue. Splines B, E, and F are presented in the second color 608b indicating that the local impedance values of the corresponding spline on catheter 300 are in the second lowest range of impedances indicating, such as more proximate to tissue than spline A.

The visualization 610 further includes a vector 616 to indicate a direction of trending impedance values. In the illustrated example, the vector 616 is oriented in the direction of the highest impedance, such as indicating the direction of cardiac tissue. In other embodiments, the vector can be oriented in the direction of lowest impedance. In one embodiment, the vector 616 can be sized on the graphical representation to correspond with the amount of impedance, such as a larger depiction of vector 616 for a larger impedance in the direction indicated by the vector. In another embodiment, the vector 616 can shaded or colored in the graphical representation to correspond with the amount of impedance, such as a color 618 of vector 616 corresponding with the color scale 606. In the illustrated example, the vector 616 is both depicted in the largest size from a plurality of available sized and in the fifth color 608e to indicate the impedance value is in the highest range of the plurality of available range of impedance values. In one embodiment, a vector can be depicted in the visualization 600 in which the impedance values are averaged to provide additional information in the graphical representation.

FIG. 6C is another example visualization 620 including a catheter model 622 of the self-expanding electroporation catheter 300. Impedances have been determined according to process 500 from the four-terminal configuration at 502. In the illustrated example, the measured impedances at 502 have been processed to obtain impedance values for each sensing electrode 350 on the splines of the electrode assembly 310. The impedance values for each sensing electrode of the electrode assembly 310 are graphically represented on the catheter model 622 of the visualization as colors 624 representative of the impedance measurement selected from the color scale 606 of multiple ranges of impedances as provided at 504. In the illustrated example visualization 620, the sensing electrodes of the catheter model 622 are represented with different colors from the color scale 606 based on the determined impedance values of the sensing electrodes. For instance, the sensing electrodes Q of catheter model 622 are presented in the first color 608a indicating that the local impedance value of the corresponding sensing electrodes 350 on catheter 300 is within the lowest range of impedances on the scale 606. Sensing electrodes R of catheter model 622 are presented in the fifth color 608e indicating that the local impedance value of the corresponding sensing electrodes 350 on catheter 300 are within the highest range of impedance on the scale 606. Sensing electrodes S of catheter model 622 are presented in the second color 608b indicating that the local impedance value of the corresponding sensing electrodes 350 on catheter 300 are within the second lowest range of impedance on the scale 606. Sensing electrodes T of catheter model 622 are presented in the third color 608c indicating that the local impedance value of the corresponding sensing electrodes 350 on catheter 300 are within the middle range of impedance on the scale 606.

FIG. 6D is another example visualization 630 including a catheter model 632 of the self-expanding electroporation catheter 300. Impedances have been determined according to process 500 from the four-terminal configuration at 502. In the illustrated example, the measured impedances at 502 have been processed to obtain impedance values for each sensing electrode 350 on the splines of the electrode assembly 310. The impedance values for each sensing electrode 350 of the electrode assembly 310 are graphically represented on the catheter model 632 of the visualization as vectors 634 representative of the impedance measurement selected from a size scale of multiple ranges of impedances and include a color from the color scale 606 of multiple ranges of impedances as provided at 504. In the illustrated example visualization 630, the vectors corresponding with sensing electrodes of the catheter model 632 are represented with different sizes and colors from the color scale 606 based on the determined impedance values of the sensing electrodes. For instance, the vectors J of catheter model 632 are presented in the smallest vectors size and first color 608a indicating that the local impedance value of the corresponding sensing electrodes 350 on catheter 300 is within the lowest range of impedances on the scale 606. Vectors K of catheter model 632 are presented in the largest vector size and fifth color 608e indicating that the local impedance value of the corresponding sensing electrodes 350 on catheter 300 are within the highest range of impedance on the scale 606. Vectors L of catheter model 622 are presented in the second smallest vector size and second color 608b indicating that the local impedance value of the corresponding sensing electrodes 350 on catheter 300 are within the second lowest range of impedance on the scale 606.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A system for ablating cardiac tissue through irreversible electroporation, the system comprising:
an electroporation and mapping catheter for performing irreversible electroporation, the catheter comprising:
a shaft having a proximal end and an opposite distal end; and
an electrode assembly extending distally from the distal end of the shaft, the electrode assembly defining a distally located central hub portion and a plurality of splines each including a distal end portion extending from the central hub portion, and a proximal end portion attached to and constrained by the outer shaft, the electrode assembly having an expanded configuration and a collapsed configuration, the electrode assembly comprising a plurality of electrodes including:
a distal ablation electrode including an ablation electrode hub portion located on the distal hub portion, and a plurality of radial segments integrally formed with the ablation electrode hub portion, each of the radial segments extending proximally along a portion of a respective one of the splines and terminating in a proximal end; and
a set of spline sensing electrodes including a plurality of sensing electrodes located on each spline;
a graphical display; and
a controller coupled to the graphical display and the catheter, the controller configured to:
determine an impedance from a four-terminal configuration, wherein a current is injected to a current carrying pair of the plurality of electrodes and a voltage is measured across a sensing pair of electrodes comprising a plurality of sensing electrodes; and
generate, on the graphical display, a graphical representation of a model of the impedance of the spline, wherein the graphical representation includes a color from a plurality of colors representative of impedance associated with each of the sensing electrodes of the plurality of sensing electrodes.

2. The system of claim 1, wherein the central hub portion includes a hub sensing electrode.

3. The system of claim 2, wherein the current carrying pair of electrodes includes the hub sensing electrode and a sensing electrode on a spline of the plurality of splines.

4. The system of any of claims 1, wherein the shaft includes a shaft electrode.

5. The system of claim 4, wherein the current carrying pair of electrodes includes the ablation electrode and the shaft electrode.

6. The system of claim 1, wherein the plurality of splines define an inner space in the expanded configuration, and the electrode assembly includes a post electrode disposed in the inner space.

7. The system of claim 6, wherein the current carrying pair of electrodes includes the post electrode.

8. The system of claim 1, the catheter further comprising a plurality of proximal ablation electrodes, each of the proximal ablation electrodes located on a respective one of the spline and having a distal end spaced from the proximal end of the adjacent radial segment of the distal ablation electrode.

9. The system of claim 1, wherein the impedance is determined for a spline from the voltage measured across the sensing pair of electrodes comprising a pair of adjacent sensing electrodes on the spline.

10. The system of claim 9, wherein the graphical representation includes a color from a plurality of colors representative of impedance associated with that spline.

11. The system of claim 1, wherein the impedance is determined from multiplexing multiple sets of sensing pairs of electrodes.

12. The system of claim 1, wherein the graphical representation includes a vector from a plurality of vectors representative of impedance associated with each of the sensing electrodes of the plurality of sensing electrodes.

13. The system of claim 1, wherein the graphical representation includes a color from a plurality of colors representative of impedance associated with each of the sensing electrodes of the plurality of sensing electrodes.

14. A system for ablating cardiac tissue through irreversible electroporation, the system comprising:
an electroporation and mapping catheter for performing irreversible electroporation, the catheter comprising:
a shaft having a proximal end and an opposite distal end; and
an electrode assembly extending distally from the distal end of the shaft, the electrode assembly defining a distally located central hub portion and a plurality of splines each including a distal end portion extending from the central hub portion, and a proximal end portion attached to and constrained by the outer shaft, the electrode assembly having an expanded configuration and a collapsed configuration, the electrode assembly comprising a plurality of electrodes including:
a distal ablation electrode including an ablation electrode hub portion located on the distal hub portion, and a plurality of radial segments integrally formed with the ablation electrode hub portion, each of the radial segments extending proximally along a portion of a respective one of the splines and terminating in a proximal end; and
a set of spline sensing electrodes including a plurality of sensing electrodes located on each spline;
a graphical display; and
a controller coupled to the graphical display and the catheter, the controller configured to:
determine an impedance from a four-terminal configuration, wherein a current is injected to a current carrying pair of the plurality of electrodes and a voltage is measured across a sensing pair of electrodes comprising a plurality of sensing electrodes; and
generate, on the graphical display, a graphical representation of a model of the impedance of that spline, wherein the graphical representation includes a color from a plurality of colors representative of impedance associated with each of the sensing electrodes of the plurality of sensing electrodes.

15. The system of claim 14, wherein the impedance is determined from multiplexing multiple sets of sensing pairs of electrodes.

16. The system of claim 15, wherein the plurality of electrodes of a sensing pair of the multiple sets of sensing pairs includes electrodes on adjacent splines.

17. The system of claim 16, wherein the graphical representation includes a vector from a plurality of vectors representative of impedance associated with each of the sensing electrodes of the plurality of sensing electrodes.

18. A system for ablating cardiac tissue through irreversible electroporation, the system comprising:
an electroporation and mapping catheter for performing irreversible electroporation, the catheter comprising:
a shaft having a proximal end and an opposite distal end; and
an electrode assembly extending distally from the distal end of the shaft, the electrode assembly defining a distally located central hub portion and a plurality of splines each including a distal end portion extending from the central hub portion, and a proximal end portion attached to and constrained by the outer shaft, the electrode assembly having an expanded configuration and a collapsed configuration, the electrode assembly comprising a plurality of electrodes including:
a distal ablation electrode including an ablation electrode hub portion located on the distal hub portion, and a plurality of radial segments integrally formed with the ablation electrode hub portion, each of the radial segments extending proximally along a portion of a respective one of the splines and terminating in a proximal end; and
a set of spline sensing electrodes including a plurality of sensing electrodes located on each spline;
a graphical display; and
a controller coupled to the graphical display and the catheter, the controller configured to:
determine an impedance from a four-terminal configuration, wherein a current is injected to a current carrying pair of the plurality of electrodes and a voltage is measured across a sensing pair of electrodes comprising a plurality of sensing electrodes; and
generate, on the graphical display, a graphical representation of a model of the impedance of the spline, wherein the graphical representation includes a color from a plurality of colors representative of impedance associated with each of the sensing electrodes of the plurality of sensing electrodes, and wherein the graphical representation includes a vector from a plurality of vectors representative of impedance associated with each of the sensing electrodes of the plurality of sensing electrodes.

19. The system of claim 18, wherein the determined impedance is applied to generate a map of the heart.

20. The system of claim 18, wherein the impedance is determined from multiplexing multiple sets of sensing pairs of electrodes.
